(19) United States
(12) Patent Application Publication
(10) Pub. No.: US 2018/0000649 A1
(43) Pub. Date: Jan. 4, 2018

(54) EARCAP AND EARPLUG

(71) Applicant: ZHUHAI ZOROSY TECHNOLOGY CO., LTD., Zhuhai (CN)

(72) Inventor: Zhongqing WANG, Zhuhai (CN)

(73) Assignee: ZHUHAI ZOROSY TECHNOLOGY CO., LTD., Zhuhai (CN)

(21) Appl. No.: 15/702,706

(22) Filed: Sep. 12, 2017

Related U.S. Application Data
(63) Continuation-in-part of application No. PCT/CN2016/084016, filed on May 31, 2016.

(51) Int. Cl. A61F 11/08; H04R 1/10

(52) U.S. Cl. CPC A61F 11/08; H04R 1/1058; H04R 1/1016; A61F 2011/085

(57) ABSTRACT

The present invention provides an earcap and an earplug, the earcap has a shell made of a flexible material, the two ends of the shell are respectively provided with a sound inlet and a sound outlet, the interior of the shell is disposed in a through manner, a filter net is disposed at a position close to the sound outlet, in the shell. The earplug comprises an earcap and a filtering core mounted in the earcap, the earcap has a shell made of a flexible material, two ends of the shell are open, the two ends of the shell are respectively provided with a sound inlet and a sound outlet, the interior of the shell is disposed in a through manner, the filtering core is mounted between the sound inlet and the sound outlet, a filter net is disposed at a position close to the sound outlet, in the shell.

### CROSS REFERENCE TO RELATED APPLICATION

This application is a continuation-in-part (CIP) of PCT Application No. PCT/CN2016/084016 filed on May 31 based on Chinese Utility Model Patent Application No. CN201520244811.6 filed on Apr. 21, 2015, the entire contents of which are incorporated herein by reference.

### TECHNICAL FIELD

The present invention relates to the field of denoising earplugs, in particular to an earplug having a denoising effect and an earcap used by such earplug.

### BACKGROUND

As the process of urbanization in China accelerates, more and more people select an outdoor life style to relieve various confronting pressures, all kinds of audio and video entertainment products that can be carried with become unnecessary assistants, correspondingly, a sound transmitting earplug is also gradually turned into an necessity in people's life.

In the other aspect, environmental noise pollution is one of three major pollution sources. With the development of industrial production, traffic transportation and urban construction and the increase of population density as well as the increase of family facilities (soundboxes, air conditioners, televisions, etc.), the environmental noise is serious day by day, and it has become one big public hazard that pollutes the social environment of humankind. Long term work under a noise environment will cause hearing loss of workers, and therefore, the workers need to adopt health protection measures.

Referring to FIG. 1, the eixsting sound transmitting earplug mainly comprises a frame body 1, a first end of the frame body 1 is a sound outlet hole 11, a second end is a lead hole 12, a lead 19 enters from the lead hole 12, a loudspeaker (not shown) is mounted in the frame body 1, the lead 19 is electrically connected to the loudspeaker, and an external sound is output to the loudspeaker by the lead 19 and is output by the loudspeaker. The existing sound transmitting earplug is further provided with an earcap 13 on the outer edge of the frame body 1, the earcap 13 is made of a flexible material, for example, made of silica gel, and the middle of the earcap 13 is through. Two ends of the earcap 13 are respectively provided with a sound inlet 16 and a sound outlet 15, a cavity body is formed between the sound inlet 16 and the sound outlet 15, and the sound outlet hole 11 of the frame body 1 can extend into the cavity body.

The existing earplug has the following defects: since the middle of the earcap 13 is through, earwax and other foreign matters in human ears will block the sound outlet hole 11 by the earcap 13 or will be attached to the loudspeaker to corrode the same by the sound outlet hole 11, sound emitting of the loudspeaker is affected, and therefore a listening effect of the earplug is affected.

The Chinese Utility Model with a patent number CN201020666901.1 discloses a noise control earplug, the earplug is provided with an earcap, i.e., a filter, and a hollow structure is disposed in the earcap. The noise control earplug can effectively weaken high frequency noise and reserve useful low and medium frequency sounds. But such earplug has the following defect: because the middle of the earcap is through, and the inner diameter of a through passage is smaller, the earwax and other foreign matters in the human ears will enter the passage and will be gradually accumulated till the passage is blocked, and consequently, the middle and low frequency sounds are attenuated, and the listening effect is affected.

The existing noise control earplug comprises an earcap, a filtering core is disposed in the earcap, and the high frequency noise is filtered by the filtering core, so as to realize a denoising purpose. However, because the existing earcap has the defect that the foreign matters easily enter the cavity body, if the foreign matters enter the cavity body, the sound outlet of the filtering core will be blocked, and as a result, the sound cannot be output.

### SUMMARY

In order to solve the problems mentioned above, a main objective of the present invention is to provide an earcap which effectively prevents foreign matters from entering a cavity body.

Another objective of the present invention is to provide an earplug ensuring that a filtering core will not be abnormally blocked.

In order to realize the main objectives mentioned above, the earcap provided by the present invention comprises a shell made of a flexible material, the two ends of the shell are open, the two ends of the shell are respectively provided with a sound inlet and a sound outlet, and the interior of the shell is disposed in a through manner, wherein a filter net is disposed at a position close to the sound outlet, in the shell. Preferably, the filter net and the shell are integrally formed.

A preferable solution is that the shell is a cylindrical shell, the sound inlet is provided with a first step part, the sound outlet is provided with a second step part, inner diameters of the first step part and the second step part are both less than the inner diameter of the middle of the shell, and the filter net is disposed on the inner side of the second step part.

An optional solution is that the shell is steplike, an outer diameter of one end of the shell close to the sound inlet is greater than an outer diameter of one end of the shell close to the sound outlet, and the outer diameter of the shell is gradually reduced from the sound inlet to the sound outlet.

A further solution is that a filtering core cavity and a sound transmitting cavity are disposed in the shell, the filtering core cavity is disposed on one side close to the sound inlet, and the sound transmitting cavity is disposed on one side close to the sound outlet.

A more further solution is that an inner diameter of the filtering core cavity is greater than that of the sound transmitting cavity, and the filtering core cavity is communicated with the sound transmitting cavity.

A more further solution is that the shell is externally provided with at least one extending piece, a first end of the extending piece is a fixed end, the fixed end is fixed on the peripheral wall of the shell, a second end of the extending piece is a free end, the free end of the extending piece extends towards a direction of the sound inlet, the extending piece is an arc slice, in an axial direction of the shell, the free end of the extending piece closest to one end of the sound inlet extends to the outer side of the sound inlet.

In order to realize the another objective mentioned above, an earplug provided by the present invention comprises an earcap and a filtering core mounted in the earcap, the earcap has a shell made of a flexible material, the two ends of the shell are open, the two ends of the shell are respectively provided with a sound inlet and a sound outlet, the interior of the shell is disposed in a through manner, and the filtering core is mounted between the sound inlet and the sound outlet, wherein a filter net is disposed at a position close to the sound outlet, in the shell.

A preferable solution is that the filtering core comprises a cylindrical shell, one end of the filtering core is provided with a sound inlet hole, the other end of the filtering core is provided with a sound outlet hole, an inner diameter of the sound inlet hole is greater than that of the sound outlet hole, the sound inlet hole is disposed towards the sound inlet, and the sound outlet hole is disposed towards the sound outlet.

A further solution is that the sound inlet hole of the filtering core is provided with a filter net film, one side of the filter net film close to the sound outlet hole is provided with a composite film, and the composite film and the filter net film are adjacently disposed.

A filter net is disposed in the earcap provided by the present invention, the filter net is disposed on one end close to the sound outlet, in this way, the filter net can effectively block foreign matters from entering into the earcap, therefore, the filtering core or an earphone disposed in the earcap can be effectively protected, and further the earplug is prevented from being blocked. Meanwhile, the filter net can protect a core element such as a loudspeaker or filter in the earplug from being polluted, the influence on an attenuation value of the sound is avoided, and therefore, a listening effect of the earplug is ensured.

Besides, both the sound inlet and the sound outlet of the earcap are provided with a step part, and the step parts can fix the filtering core in the earcap, and avoid the falling of the filtering core from the earcap.

Furthermore, the filtering core cavity and the sound transmitting cavity are disposed in the earcap, the filtering core can be mounted in the filtering core cavity, the inner diameter of the filtering core cavity is larger, while the inner diameter of the sound transmitting cavity is smaller, and the inner diameter of the sound transmitting cavity is less than the outer diameter of the filtering core, thus the filtering core is prevented from moving into the sound transmitting cavity.

In addition, the extending piece is disposed on the peripheral wall of the earcap, and can increase friction between the earcap and an ear canal, and avoid falling of the earcap from the ear canal.

Besides, the filtering core disposed in the earcap has the filter net film and the composite film, which can effectively filter a high frequency noise.

### BRIEF DESCRIPTION OF THE DRAWINGS

FIG. 1 is a structural schematic diagram of the existing earphone and earcap.

FIG. 2 is a structural schematic diagram of a first embodiment of an earplug of the present invention.

FIG. 3 is a structural schematic diagram of an earphone and a first embodiment of an earcap of the present invention.

FIG. 4 is a structural schematic diagram of a second embodiment of an earplug of the present invention.

FIG. 5 is a structural schematic diagram of an earphone and a second embodiment of an earcap of the present invention.

FIG. 6 is a section view of a third embodiment of an earplug of the present invention.

FIG. 7 is a structural exploded view of the third embodiment of the earplug of the present invention.

FIG. 8 is a structural diagram of a fourth embodiment of an earplug of the present invention.

FIG. 9 is a section view of a fifth embodiment of an earplug of the present invention.

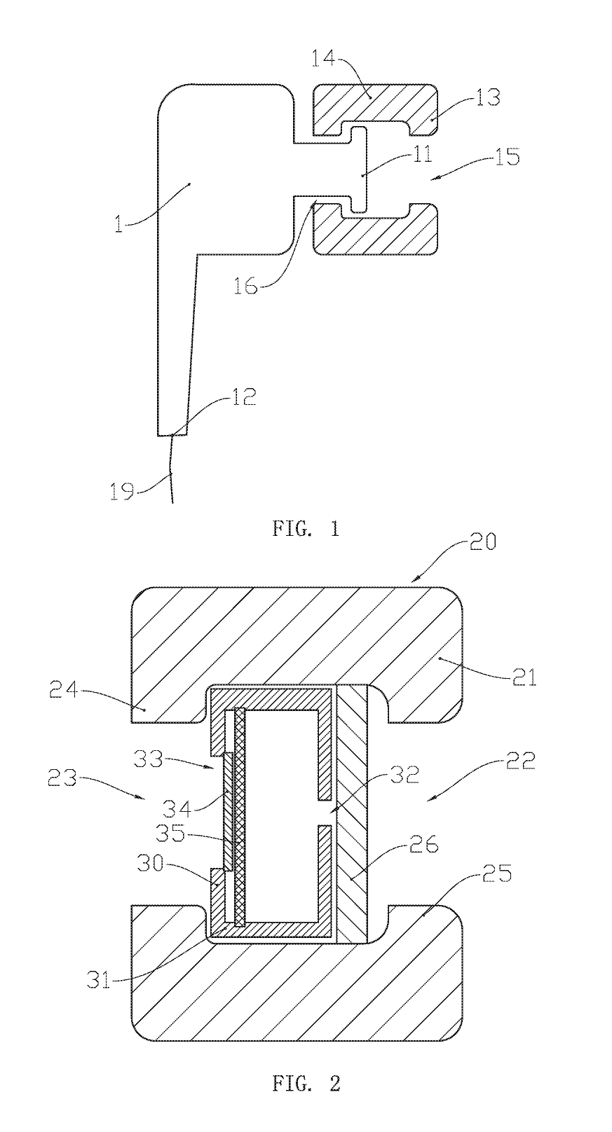

The present invention is further explained in combination with drawings and embodiments.

### DETAILED DESCRIPTION

An earcap of the present invention can cover an earphone, a filtering core also can be mounted in the earcap to form an earplug, if the earcap is mounted on the earphone, a sound transmitting earplug can be formed, and if the filtering core is mounted in the earcap, a denoising earplug can be formed.

### First Embodiment

Referring to FIG. 2, the earplug of the present embodiment is a denoising earplug, which comprising an earcap 20 and a filtering core 30 mounted in the earcap 20, the earcap 20 has a shell 21 which is a hollow cylinder, two ends of the earcap 20 are respectively provided with a sound inlet 23 and a sound outlet 22, the part between the sound inlet 23 and the sound outlet 22 is disposed in a through manner, that is, air can penetrate through a space between the sound inlet 23 and the sound outlet 22.

One end of the shell 21 close to the sound inlet 23 is provided with a step part 24, and one end close to the sound outlet 22 is also provided with a step part 25. From FIG. 2, it can be seen that the step part 24 extends towards a central axial direction of the shell 21 from the inner wall of the shell 21, that is, extends towards an interior direction of the shell 21 from the inner wall of the shell 21, therefore, an inner diameter of the step part 24 is less than that of the middle of the shell 21. Similarly, the step part 25 also extends towards the interior direction of the shell 21 from the inner wall of the shell 21, and the inner diameter of the step part 25 is less than that of the shell 21.

One side close to the sound outlet 22 is provided with a filter net 26, from FIG. 2, it can be seen that the filter net 26 is disposed on one side of the step part 25 close to the middle of the shell 21, a peripheral wall of the filter net 26 is fixed on the inner wall of the shell 21, and an outer diameter of the filter net 26 is greater than the inner diameter of the step part 25, therefore, falling of the filter net 26 from the shell 21 is avoided.

In the present embodiment, the shell 21 is formed by performing injection molding on a flexible material such as silica gel, besides, the filter net 26 and the shell 21 are integrally molded, therefore, when the earcap 20 is manufactured, liquid silica gel or a soft material is injected into a die at one step, and the shell 21 and the filter 26 are formed at one step.

A cavity is formed in the earcap 21, the filtering core 30 is mounted in the cavity, and specifically, the filtering core 30 is mounted between the step part 24 and the filter net 26. From FIG. 2, it can be seen that the filtering core 30 has a shell 31, the shell 31 can be made of plastic or silica gel, etc., two ends of the shell 31 are respectively provided with a sound inlet hole 33 and a sound outlet hold 32, the sound inlet hole 33 is disposed towards the sound inlet 23 of the earcap 20, and the sound outlet hole 32 is disposed towards the sound outlet 22 of the earcap 20.

The inner diameter of the sound inlet hole 33 is greater than that of the sound outlet hold 32, but the inner diameter of the sound inlet hole 33 is less than that of the sound inlet 23. A filter net film 34 is disposed at a position close to the sound inlet hole 33, and one side of the filter net film 34 close to the sound outlet hole 32 is provided with a composite film 35, preferably, the composite film 35 is a resin film, such as a PE film or a PET film.

Besides, the filter net film 34 and the composite film 35 are disposed adjacently, and a diameter of the composite film 35 is greater than that of the filter net film 34. In the present embodiment, the filter net film 34 and the composite film 35 are both fixed at a position of the shell 31 close to the sound inlet hole 33, and both the filter net film 34 and the composite film 35 cannot move relative to the shell 31.

The sound enters the earcap 20 from the sound inlet 23 and then passes by the filtering core 30 and enters an ear canal from the sound outlet 22, since the inner diameter of the sound inlet hole 33 of the filtering core 30 is greater than that of the sound outlet hole 32, after the sound enters the filtering core 30, the noise of a high frequency part cannot penetrate through the sound outlet hole 32, therefore, the high frequency noise is filtered. Besides, the filter net film 34 and the composite film 35 are disposed on the filtering core 30, the sounds of respective frequency bands can be attenuated, in this way, it can be ensured that an intermediate signal that can be accepted by the human ears can effectively penetrate through the sound outlet hole 32, the purpose that people wearing such earplug can hear the speaking sound of other people in a case of emergency is ensured, and the safety of a wearer is ensured.

In the present embodiment, the filter net film 34 can be nonwoven cloth made of a dacron or nylon material matched according to different thicknesses and colors and textures, and can also be of a woolen cloth net film, and therefore, according to actual needs, the loudness of the sound and frequency can be adjusted by controlling a mesh number and textures of the filter net film 34 and the size of a vent hole. Specifically, the less the mesh number of the filter net film 34 is, the larger a ventilation property is, and the larger the sound is, conversely, the more the mesh number of the filter net film 34 is, the smaller the ventilation property is, and the smaller the sound is. Besides, the size of the vent hole is mainly used for frequency attenuation except for certain adjusting action on the sound. Therefore, after the diameter of the filter net film 34 is determined, the present embodiment can adjust the size of the vent hole by a back adhesive process, thus, if the outer diameter of the filter net film 34 is unchanged, the smaller the inner diameter is, the smaller the ventilation property is, the higher the frequency specific to the filter net film 34 is, and the more attenuated a sound signal of the corresponding frequency is. Conversely, if the inner diameter of the filter net film 34 is larger and the ventilation property is larger, the frequency specific to the filter net film 34 is correspondingly reduced, and the attenuation of a high frequency signal is also reduced.

The composite film 35 can be made of a PET film, the PET film has well better high temperature resistance and high toughness and airtightness, therefore, the PET film used by the present embodiment is mainly used for sound processing, an action thereof for the sound is mainly embodied in the medium and low frequency bands with the frequency from 500 Hz to 2000 Hz, and the roles of filtering sound impurity and keeping a sound wave curve stable are played.

With the higher and higher requirements of people on a sound quality and increasing progress of technology, regular textures with different shapes can be made on the surface of the PET film according to needs by plating and etching molding technologies, these tiny textures can be combined with the structure of a cavity body of the filter, and play roles of reflection, scattering, diffraction and attenuation on the sound, and then a desired sound quality can be obtained. Except for the processes above, a titanizing process can also be used, i.e., plating is performed on two surfaces of the PET film to form a layer of titanium film. The titanizing can increase rigidity of the PET film and improve a high frequency response speed, the sound is thorough and clear, and the sensitivity is higher, such that people can well hear a human voice even in a noisy environment. Besides, the textures formed by etching are merely formed on one side of the PET film close to the sound inlet hole 33, i.e., no textures are disposed on one side close to the sound outlet hole 32.

Of course, the composite film 35 can be made of a silica gel film, which has very high temperature resistance, oil dirty resistance and chemical corrosion resistance, and has better acoustic performance. Because a thickness of the silica gel film is very important to the sound, in the present embodiment, the silica gel film is made into the thickness of 0.03 mm or even 0.01 mm by a liquid silica gel injection process. Besides, in the present embodiment, accurate location can be realized on the silica gel film by high precision of a laser technology, nine small holes are formed in the silica gel film and are arrayed in a manner of 3*3, and each small hole has a diameter of only 0.006 mm. That is to say, the small holes in the silica gel film can be formed in a manner that laser penetrates through the silica gel film.

By controlling the thickness of the silica gel film and the precision of an aperture of the silica gel film, the frequency and loudness of the sound can be effectively adjusted, specific frequencies harmful to hearing are reduced, and the medium and low frequency useful signals can be reserved, so as to achieve an attenuation effect.

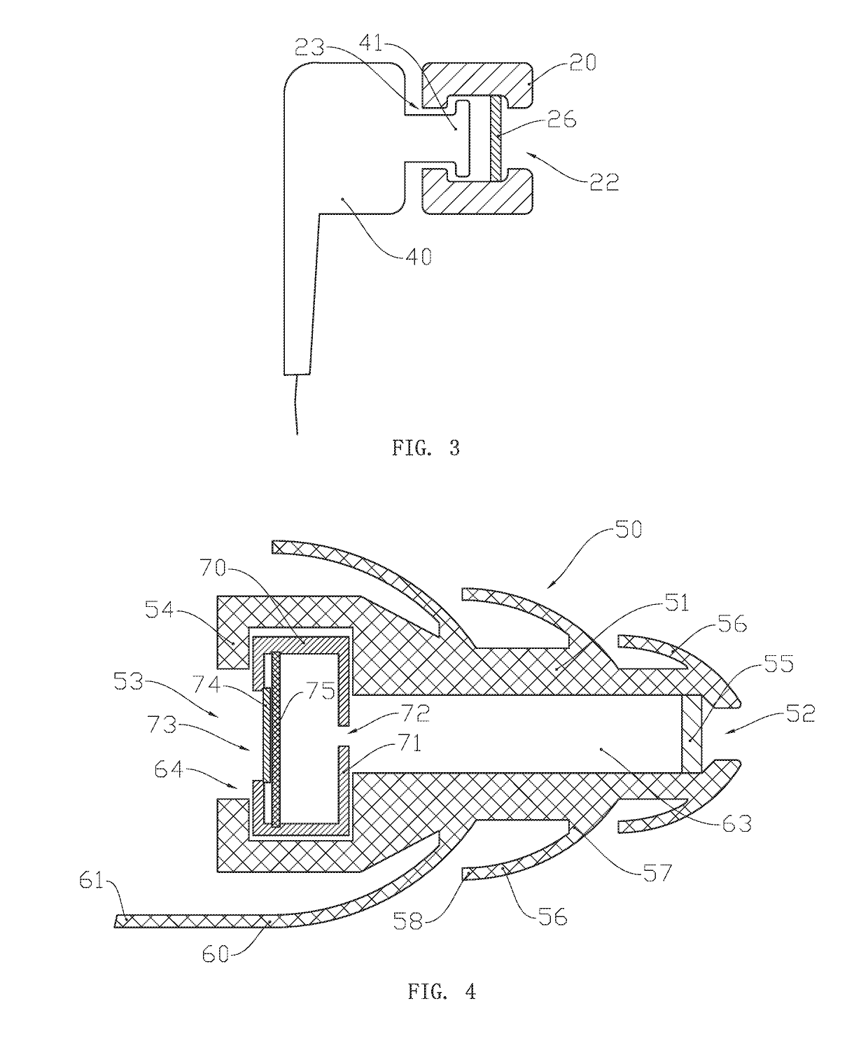

FIG. 2 shows a denoising earplug, the earcap 20 of the present embodiment can also be applied to the sound transmitting earplug, as shown in FIG. 3, the sound transmitting earplug has an earcap 20, the part between a sound inlet 23 and a sound outlet 22 of the earcap 20 is disposed in a through manner, that is, air can penetrate through a space between the sound inlet 23 and the sound outlet 22. One side of the earcap 20 close to the sound outlet 22 is provided with a filter net 26, and the filter net 26 and a shell of the earcap 20 can be integrally formed.

The earplug further comprises a frame body 40 of one earphone, and one end of the frame body 40 is provided with a sound outlet hole 41, which extends into the earcap 20. For example, a circuit board can be disposed in the frame body 40, the circuit board is provided with a loudspeaker, the loudspeaker receives an audio signal which enters the earcap 20 by the sound outlet hole 41, and the sound passes by the filter net 26, and then enters the ear canal of a human body from the sound outlet 22.

Since a filter net 26 is disposed in the earcap 20, foreign matters such as earwax and the like cannot enter the earcap, and thus will not block the filtering core or the sound outlet hole of the frame body, an influence on the filtering core or a device in the frame body is avoided, and normal work of the earplug is ensured.

### Second Embodiment

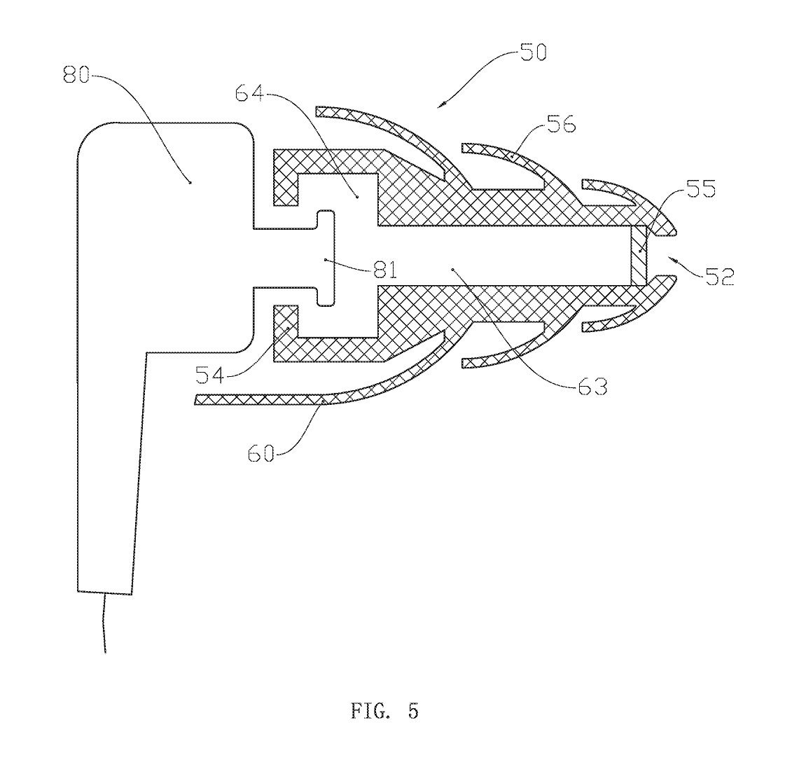

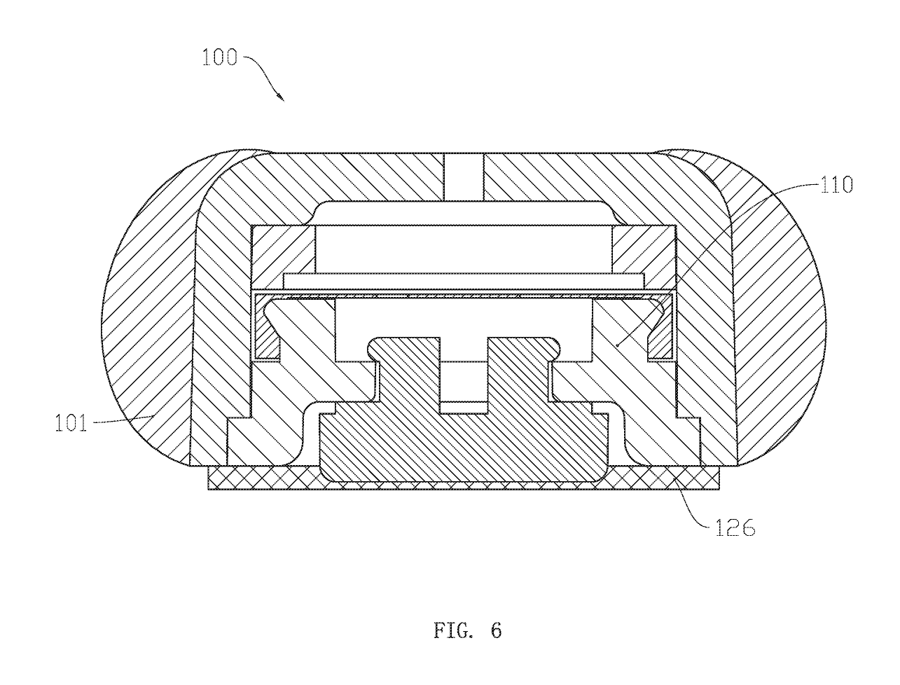

The earplug of the present embodiment can be a denoising earplug as shown in FIG. 4, and can also be a sound transmitting plug as shown in FIG. 5, the denoising earplug has an earcap 50, a filtering core 70 is disposed in the earcap 50, while the earcap 50 of the sound transmitting earplug is matched with a frame body 80 of the earphone.

Referring to FIG. 4, the earcap 50 of the denoising earplug has a steplike shell 51 made of a flexible material, such as silica gel. The shell 51 has a plurality of sections of cylinders with unequal outer diameters, so as to form a step shape. Two ends of the earcap 50 are open and are respectively provided with a sound inlet 53 and a sound outlet 52, an inner diameter of the sound inlet 52 is greater than that of the sound outlet 52, a step part 54 is disposed at a position close to the sound inlet 53, and the step part 54 extends towards an axial direction of the shell 51 from the inner wall of the shell 51. Besides, an outer diameter of the shell 51 on one end of the sound inlet 53 is greater than that on one end of the sound outlet 52, and the outer diameter of the shell 51 is gradually reduced from the end of the sound inlet 53 to the end of the sound outlet 52.

A plurality of extending pieces 56 are disposed on the outer part of the shell 51, a first end of each extending piece 56 is a fixing end 57, the fixing end 57 is fixed on the peripheral wall of the shell 51, a second end of each extending piece 56 is a free end 58, and the free end 58 of each extending piece 56 extends towards a direction of the sound inlet 53. Preferably, the extending pieces 56 are all arc slices, and the extending pieces 56 and the shell 51 are integrally formed by injection molding, therefore, the extending pieces 56 are also formed by performing injection molding on a flexible material such as silica gel.

Besides, the free end 61 of an extending piece 60 closest to the sound inlet 53 extends to exceed the sound outlet 53, that is, extends to exceed the outer side of the sound outlet 53. In this way, after a wearer wears the earcap 50, even though most part of the earcap 50 is located in the ear canal, the free end 61 of the extending piece 60 is stilled located on the outer side of the ear canal, and it is convenient for the wearer to take down the earcap 50.

A filter net 55 is disposed on one side of the sound outlet 52, the filter net 55 is located in the shell 51, and the filter net 55 and the shell 51 can be integrally formed, for example, when the shell 55 and the filter net 55 are manufactured, the filter net 55 and the shell 51 can be formed at one step by injecting liquid silica gel or soft material into a die.

A filtering core cavity 64 and a sound transmitting cavity 63 are disposed in the earcap 50, the filtering core cavity 64 is disposed on one end close to the sound inlet 53, the sound transmitting cavity 63 is disposed on one end close to the sound outlet 52, and the filtering core cavity 64 and the sound transmitting cavity 63 are communicated with each other. The filtering core 70 is mounted in the filtering core cavity 64, from FIG. 4, it can be seen that an inner diameter of the filtering core cavity 64 is greater than that of the sound transmitting cavity 63, and an outer diameter of the filtering core 70 is greater than the inner diameter of the sound transmitting cavity 63, thus the filtering core 70 is prevented from moving into the sound transmitting cavity 63. Preferably, an outer diameter of the filtering core 70 is slightly greater than the inner diameter of the filtering core cavity 64, that is, the filtering core 70 and the shell 51 are in interference fit, therefore, it can be ensured that the filtering core 70 is fixed in the filtering core cavity 64.

The filtering core 70 has a shell 71, the shell 71 can be made of plastic or silica gel, two ends of the shell 71 are respectively provided with a sound inlet hole 73 and a sound outlet hole 72, the sound inlet hole 73 is disposed towards the sound inlet 53 of the earcap 50, and the sound outlet hole 72 is disposed towards the sound outlet 52 of the earcap 50.

The inner diameter of the sound inlet hole 73 is lager than that of the sound outlet hole 72, but the inner diameter of the sound inlet hole 73 is less than that of the sound inlet 53. A filter net film 74 is disposed at a position close to the sound inlet hole 73, a composite film 75 is disposed on one side of the filter net film 74 close to the sound outlet hole 72, preferably, the composite film 75 is a resin film, for example, a PE film or PET film. Besides, the filter net film 74 and the composite film 75 are adjacently disposed, and the diameter of the composite film 75 is greater than that of the filter net film 74. In the present embodiment, the filter net film 74 and the composite film 75 are both fixed at a position close to the sound inlet hole 73, and both the filter net film 74 and the composite film 75 cannot move relative to the shell 71.

In the present embodiment, the filter net film 74 can be nonwoven cloth made of a dacron or nylon material matched according to different thicknesses and colors and textures, and can also be of a woolen cloth net film, and therefore, according to actual needs, the loudness of the sound and frequency can be adjusted by controlling a mesh number and textures of the filter net film 74 and the size of a vent hole. Specifically, the less the mesh number of the filter net film 74 is, the larger a ventilation property is, and the larger the sound is, conversely, the more the mesh number of the filter net film 74 is, the smaller the ventilation property is, and the smaller the sound is. Besides, the size of the vent hole is mainly used for frequency attenuation except for certain adjusting action on the sound. Therefore, after the diameter of the filter net film 74 is determined, the present embodiment can adjust the size of the vent hole by a back adhesive process, thus, if the outer diameter of the filter net film 74 is unchanged, the smaller the inner diameter is, the smaller the ventilation property is, the higher the frequency specific to the filter net film 74 is, and the more attenuated a sound signal of the corresponding frequency is. Conversely, if the inner diameter of the filter net film 74 is larger and the ventilation property is larger, the frequency specific to the filter net film 74 is correspondingly reduced, and the attenuation of a high frequency signal is also reduced.

The composite film 75 can be made of a PET film, the PET film has well high temperature resistance and high toughness and airtightness, therefore, the PET film used by the present embodiment is mainly used for sound processing, an action thereof for the sound is mainly embodied in the medium and low frequency bands with the frequency from 500 Hz to 2000 Hz, and the roles of filtering sound impurity and keeping a sound wave curve stable are played.

With the higher and higher requirements of people on a sound quality and increasing progress of technology, regular textures with different shapes can be made on the surface of the PET film according to needs by plating and etching molding technologies, these tiny textures can be combined with the structure of a cavity body of the filter, and play roles of reflection, scattering, diffraction and attenuation on the sound, and then a desired sound quality can be obtained. Except for the processes above, a titanizing process can also be used, i.e., plating is performed on two surfaces of the PET film to form a layer of titanium film. The titanizing can increase rigidity of the PET film and improve a high frequency response speed, the sound is thorough and clear, and the sensitivity is high, such that people can well hear a human voice even in a noisy environment. Besides, the textures formed by etching are merely formed on one side of the PET film close to the sound inlet hole 73, i.e., no textures are disposed on one side close to the sound outlet hole 72.

Of course, the composite film 75 can be made of a silica gel film, which has very high temperature resistance, oil dirty resistance and chemical corrosion resistance, and has better acoustic performance. Because a thickness of the silica gel film is very important to the sound, in the present embodiment, the silica gel film is made into the thickness of 0.03 mm or even 0.01 mm by a liquid silica gel injection process. Besides, in the present embodiment, accurate location can be realized on the silica gel film by high precision of a laser technology, nine small holes are formed in the silica gel film and are arrayed in a manner of 3*3, and each small hole has a diameter of only 0.006 mm. That is to say, the small holes in the silica gel film can be formed in a manner that laser penetrates through the silica gel film.

By controlling the thickness of the silica gel film and the precision of an aperture of the silica gel film, the frequency and loudness of the sound can be effectively adjusted, specific frequencies harmful to hearing are reduced, and the medium and low frequency useful signals can be reserved, so as to achieve an attenuation effect.

The sound enters the earcap 50 from the sound inlet 53 and then passes by the filtering core 70 and enters an ear canal from the sound outlet 52, since the inner diameter of the sound inlet hole 73 of the filtering core 70 is greater than that of the sound outlet hole 72, after the sound enters the filtering core 70, the noise of a high frequency part cannot penetrate through the sound outlet hole 72, therefore, the high frequency noise is filtered. Besides, the filter net film 74 and the composite film 75 are disposed on the filtering core 30, the sounds of respective frequency bands can be attenuated, in this way, it can be ensured that an intermediate signal that can be accepted by the human ears can effectively penetrate through the sound outlet hole 72, the purpose that people wearing such earplug can hear the speaking sound of other people in a case of emergency is ensured, and the safety of a wearer is ensured.

As shown in FIG. 5, the sound transmitting earplug also has an earcap 50 and is provided with a frame body 80 of the earphone, and one end of the frame body 80 is provided with a sound outlet hole 81, which extends into a filtering core cavity 64 of the earcap 50. For example, a circuit board can be disposed in the frame body 80, the circuit board is provided with a loudspeaker, the loudspeaker receives an audio signal which enters the earcap 50 by the sound outlet hole 81, and the sound passes by the filter net 55, and then enters an ear canal of a human body from the sound outlet 52.

The filter net is disposed in the earcap of the present invention and foreign matters are prevented from entering the earcap, so that the filtering core is prevented being blocked or the device such as a loudspeaker in the earphone is prevented from being influenced. Besides, the filter net film and the composite film are disposed in the filtering core and can attenuate the sounds of respective frequency bands, the filtering core has a capacity of filtering high frequency noise per se, after the sound passes by the filtering core, not only is the high frequency noise filtered, but also the excessive attenuation of a medium frequency signal in speaking of people is avoided, therefore, it can be ensured that a wearer wearing the earplug in a case of emergency can hear the speaking sound of people.

Of course, during actual application, the present invention has many changes, for example, the shell of the earcap can be made of other flexible materials; or the shapes of the earcap and the filtering core can be designed into other shapes, and such change can also realize the objectives of the present invention.

### Third Embodiment

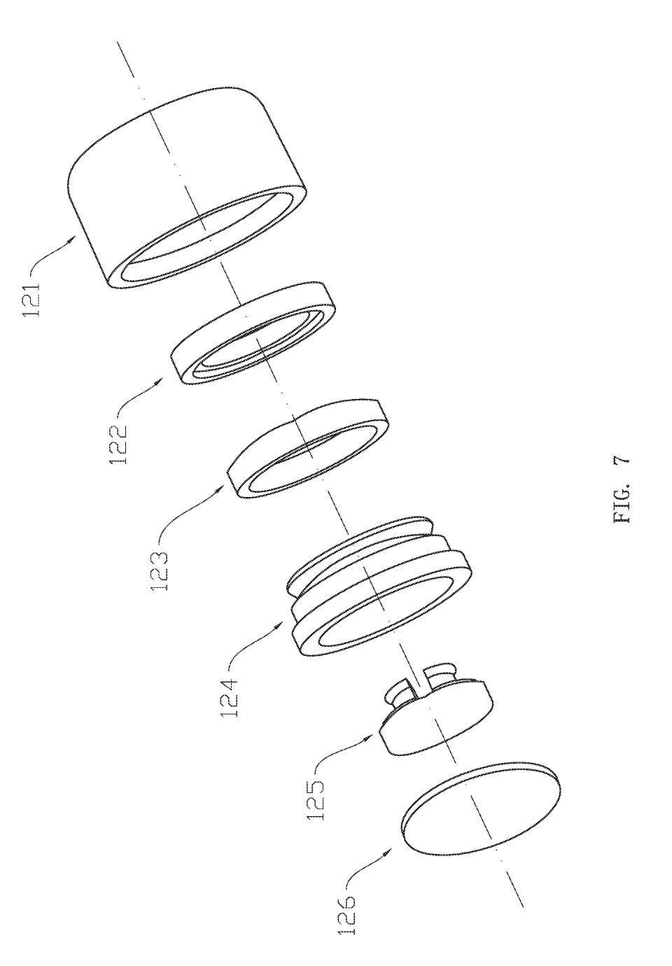

Referring to FIGS. 6 and 7, an earplug 100 of the present embodiment comprises an earplug body 101 and a filtering core 110, the filtering core 110 is disposed in the earplug body 101, the filtering core 110 comprises a cover body 121, a fixing element 122, a high frequency filtering part 123, a fixing sleeve 124, a dustproof part 125 and a low frequency filtering part 126 from top to bottom in sequence, wherein the dustproof part 125 can be same as the foregoing filter net in structure, and is not repeated.

### Fourth Embodiment

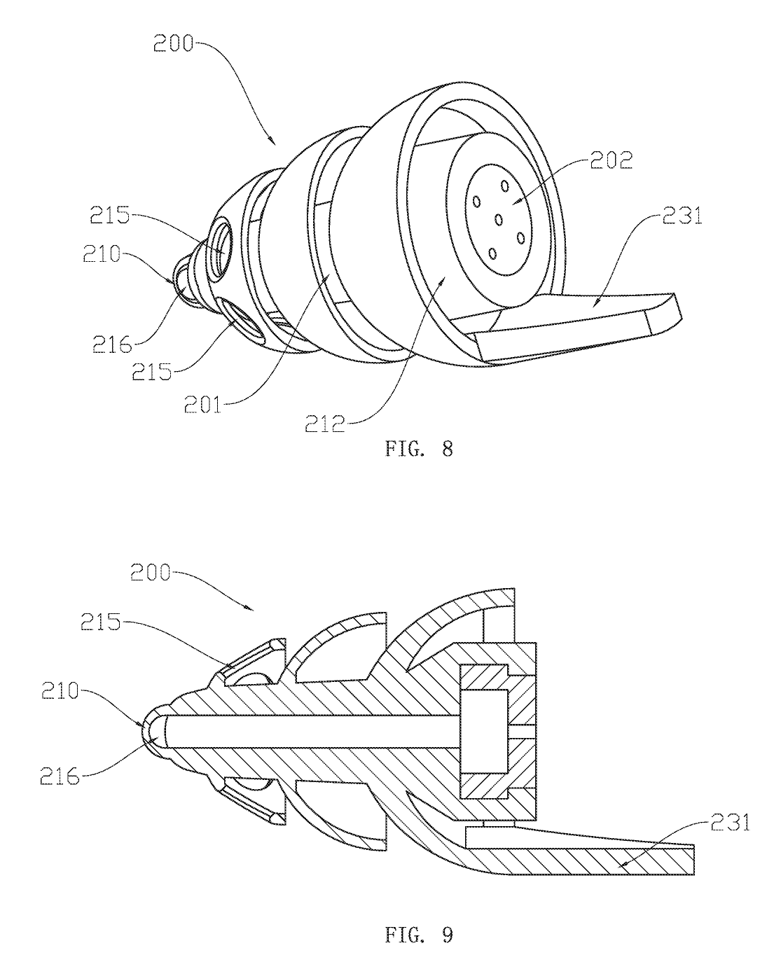

Referring to FIGS. 8 and 9, the earplug 200 of the present embodiment comprises a frame body 201 and a filter 202; the filter 202 is assembled in the frame body 201, the earplug 200 is provided with a bulge 210, the bulge 210 is located on one end away from a main body 212 of the filter 202, a cleaning hole 216 is formed between the bulge 210 and the main body 212, and an arc piece is fixedly connected on the outer wall of the main body 212.

In the present embodiment, an air pressure hole 215 is disposed in the arc piece and penetrates through the arc piece, and a holding part 231 is fixedly connected on the arc piece. The air pressure hole 215 can enable an ear canal pressure to be balanced with outside pressure, and the holding part 231 extends along a direction from left to right from the arc piece, such that a user can conveniently hold the earplug 200.

The earplug of the present invention can serve as a sound transmitting earplug or denoising earplug for use, is used under a relative noisy environment particularly, and is suitable for working staff in occasions such as construction sites, mine fields, etc., such that environment noise is reduced, and an influence on physical and psychological health of the staff caused by long term working under a high noise environment is avoided.

1. An earcap, comprising:
a shell made of a flexible material, wherein two ends of the shell are open, the two ends of the shell are respectively provided with a sound inlet and a sound outlet, and an interior of the shell is disposed in a through manner;
wherein,
a filter net is disposed at a position close to the sound outlet, in the shell.

2. The earcap according to claim 1, wherein,
the shell is a cylindrical shell, the sound inlet is provided with a first step part, the sound outlet is provided with a second step part, inner diameters of both the first step part and the second step part are less than an inner diameter of the middle of the shell, and the filter net is disposed on an inner side of the second step part.

3. The earcap according to claim 1, wherein,
the shell is step-like, an outer diameter of one end of the shell close to the sound inlet is greater than an outer diameter of one end of the shell close to the sound outlet, and an outer diameter of the shell is gradually reduced from the sound inlet to the sound outlet.

4. The earcap according to claim 3, wherein,
a filtering core cavity and a sound transmitting cavity are disposed in the shell, the filtering core cavity is disposed on one side of the shell close to the sound inlet, and the sound transmitting cavity is disposed on one side of the shell close to the sound outlet.

5. The earcap according to claim 4, wherein,
an inner diameter of the filtering core cavity is greater than an inner diameter of the sound transmitting cavity.

6. The earcap according to claim 4, wherein,
the filtering core cavity is communicated with the sound transmitting cavity.

7. The earcap according to claim 4, wherein,
one end of the shell close to the sound inlet is provided with a third step part, and an inner diameter of the third step part is less than an inner diameter of the filtering core cavity.

8. The earcap according to claim 3, wherein,
the shell is externally provided with at least one extending piece, a first end of the extending piece is a fixed end, the fixed end is fixed on a peripheral wall of the shell, a second end of the extending piece is a free end, and the free end of the extending piece extends toward a direction of the sound inlet.

9. The earcap according to claim 8, wherein,
the extending piece is an arc slice.

10. The earcap according to claim 8, wherein,
in an axial direction of the shell, the free end of the extending piece closest to one end of the sound inlet extends to an outer side of the sound inlet.

11. The earcap according to claim 1, wherein,
the filter net and the shell are integrally formed.

12. An earplug, comprising:
an earcap and a filtering core mounted in the earcap;
wherein the earcap has a shell made of a flexible material, two ends of the shell are open, the two ends of the shell are respectively provided with a sound inlet and a sound outlet, an interior of the shell is disposed in a through manner, and the filtering core is mounted between the sound inlet and the sound outlet;
wherein,
a filter net is disposed at a position close to the sound outlet, in the shell.

13. The earplug according to claim 12, wherein,
the filtering core comprises a cylindrical shell, one end of the filtering core is provided with a sound inlet hole, an other end of the filtering core is provided with a sound outlet hole, an inner diameter of the sound inlet hole is greater than an inner diameter of the sound outlet hole, the sound inlet hole is disposed toward the sound inlet, and the sound outlet hole is disposed toward the sound outlet.

14. The earplug according to claim 12, wherein,
the sound inlet hole of the filtering core is provided with a filter net film, one side of the filter net film close to the sound outlet hole is provided with a composite film, and the composite film and the filtering net film are adjacently disposed.

15. The earplug according to claim 14, wherein,
the composite film is a resin film.

16. The earplug according to claim 14, wherein,
the filter net film is nonwoven cloth made of dacron or nylon, or is a woolen cloth net film.

17. The earplug according to claim 15, wherein,
the composite film is a PET film.

18. The earplug according to claim 17, wherein,
a plurality of regular textures are formed on a surface of the PET film or a titanium film is plated on the PET film.

19. The earplug according to claim 15, wherein,
the composite film is a silica gel film, a thickness of the silica gel film is less than 0.03 mm, and a plurality of small holes are formed in the silica gel film.

20. The earplug according to claim 19, wherein,
the silica gel film is made by a liquid silica gel injection process, and the plurality of small holes are made in a manner that laser penetrates through the silica gel film.
